SEQUENCE LISTING (13 sequences)

SEQ ID NO: 1 - DNA, 17 bp, Artificial Sequence
  Forward Primer
gtgctatctggagtcct

SEQ ID NO: 2 - DNA, 16 bp, Artificial Sequence
  Reverse Primer
ctgttgttgccagcaa

SEQ ID NO: 3 - DNA, 17 bp, Artificial Sequence
  Tag Man Probe
catcacctacttcaact

SEQ ID NO: 4 - DNA, 24 bp, Artificial Sequence
  Forward Primer
ctctgcttcctctaggaacaacag

SEQ ID NO: 5 - DNA, 20 bp, Artificial Sequence
  Reverse Primer
tgttggcgatcttcagctca

SEQ ID NO: 6 - DNA, 21 bp, Artificial Sequence
  Tag Man Probe
agttccagatcacatcgagtt

SEQ ID NO: 7 - DNA, 19 bp, Artificial Sequence
  Forward Primer
gaaggtgaaggtcggagtc

SEQ ID NO: 8 - DNA, 20 bp, Artificial Sequence
  Reverse Primer
gaagatggtgatgggatttc

SEQ ID NO: 9 - DNA, 22 bp, Artificial Sequence
  Tag Man Probe
aggctgagaacgggaagcttgt

SEQ ID NO: 10 - DNA, 21 bp, Artificial Sequence
  SiRNA Target Sequence
acatgaacttggtgttggctt

SEQ ID NO: 11 - DNA, 65 bp, Artificial Sequence
  Sense Oligonucleotide for SiRNA
gatcccggccaacaccaagttcatgtttcaagagaacatgaacttggtgttggcttttttggaaa

SEQ ID NO: 12 - DNA, 21 bp, Artificial Sequence
  SiRNA Target Sequence
aagccaacaccaagttcatgt

SEQ ID NO: 13 - DNA, 65 bp, Artificial Sequence
  Antisense Oligonucleotide for SiRNA
agcttttccaaaaaagccaacaccaagttcatgttctcttgaaacatgaacttggtgttggccgg

(19) United States
(12) Patent Application Publication
(10) Pub. No.: US 2005/0215509 A1
(43) Pub. Date: Sep. 29, 2005

(54) Method for inhibiting jnk-1 kinase activity by SCCA

(75) Inventors: Chika Katagirl, Yokohama-shi (JP); Jotaro Nakanishi, Yokohama-shi (JP); Toshihiko Hibino, Yokohama-shi (JP)

Correspondence Address: SNIDER & ASSOCIATES, P. O. BOX 27613, WASHINGTON, DC, 20038-7613, US

(73) Assignee: Shiseido Co., Ltd., Tokyo (JP)

(21) Appl. No.: 11/087,804

(22) Filed: Mar. 24, 2005

(30) Foreign Application Priority Data
Mar. 26, 2004 (JP) 2004-093209

(51) Int. Cl. A61K 48/00

(52) U.S. Cl. 514/44

(57) ABSTRACT

The present invention provides a method and composition that inhibit the kinase activity of JNK-1 (c-Jun N-terminal kinase 1) in cells by increasing the expression of squamous cell carcinoma antigen (SCCA) in cells.

### BACKGROUND OF THE INVENTION

1. Field of the Invention

The present invention provides a method for inhibiting the activity of JNK-1 by increasing the expression of squamous cell carcinoma antigen (SCCA).

2. Prior Art

Apoptosis refers to cell death that is brought about for the purpose of eliminating unnecessary cells during the course of development, removing cells in order to maintain homeostasis or defending the body against infections and cancer. Apoptosis is known to be mediated by various signal transmission systems. Apoptosis induction signals are transmitted to control factors involved in the induction or inhibition of apoptosis in cells, and when cell death is determined by these factors, apoptosis factors are ultimately activated resulting in induction of cell death. Apoptosis induction factors are triggered by various types of stress such as ultraviolet (UV) radiation, exposure to oxidizing agents and drying, and cell death is determined as a result of signals being transmitted to various apoptosis control factors such as factors of the Bc1-2 family, various caspases, JNK and p53.

JNK (c-Jun N-terminal kinase) is a type of apoptosis control factor that belongs to the family of MAP kinases that play a central role in information transmission during cell reproduction, differentiation and canceration. This JNK is also activated in response to various stress stimuli. There are three genes for JNK consisting of JNK-1, JNK-2 and JNK-3. In contrast to JNK-1 and JNK-2 being present primarily in epidermic cells, JNK-3 is known to be present in the brain. In contrast to JNK-1 inducing apoptosis by being activated by ultraviolet radiation, JNK-2 is not activated by ultraviolet radiation, and the two are known to have different functions (Hochealinger K. et al., Oncogene 21, 2441-2445, Butterfield L. et al., J. Biol. Chem. 272(15), 10110-10116, 1997). The known endogenous inhibitory factor of JNK is JNK interacting protein (JIP) (M. Dickens et al., Science, Vol. 277, p. 693 (1997); A. J. Whitmarsh et al., Science, Vol. 281, p. 1671 (1998)). However, JIP is known to have low specificity and bind with these JNK as well as other MAP kinases involved in upstream activation. Being able to specifically inhibit the activation of JNK would-make it possible to inhibit apoptosis induced by JNK, thereby leading to expectations of the development of new treatment and prevention methods, cosmetics and pharmaceutical compositions for various disorders caused by ultraviolet radiation and other stress stimuli, and particularly skin disorders.

### DISCLOSURE OF THE INVENTION

The object of the present invention is to elucidate the mechanism of apoptosis of cells, and particularly keratinocytes, and provide a novel and effective anti-apoptosis means.

Psoriasis is a chronic inflammatory skin disorder affecting approximately 2% of the European population. (Koo J. Dermatol. Clin., 1996, 14: 485-496). It is characterized by infiltration of the skin by activated T-cells and hyperplastic regenerative epidermal growth (Gottlieb A. B. et al., J. Invest. Dermatol. 1992, 98: 302-309). Psoriasis is believed to occur due to the involvement of various environmental factors in addition to genetic factors. When epidermis in which psoriasis has occurred is compared with normal epidermis, increased expression of squamous cell carcinoma antigen (SCCA) is known to be observed in the upper layer of the psoriatic epidermis (Takeda A. et al., J. Invest. Dermatol. (2002) 118(1), 147-154). SCCA is an antigen that was discovered in cervical squamous cell carcinoma cells, demonstrates high blood concentrations in squamous cell carcinoma of the cervix, lungs, esophagus and skin, and is frequently used to diagnose and assess the effects of treatment of squamous cell carcinoma. Although concentration of SCCA in blood is also used to diagnose epidermal squamous cell carcinoma, in research conducted by the inventors of the present invention, expression of SCCA was observed to not be elevated in squamous cell carcinoma of the epidermis. SCCA is encoded by two genes consisting of SCCA-1 and SCCA-2 arranged in tandem on chromosome 18q21.3. Both SCCA-1 and SCCA-2 are proteins having a molecular weight of about 45,000, and although they have an extremely high degree of homology, their amino acid sequences differ at the reaction site, and are thought to demonstrate different functions for this reason (Schick et al., J. Biol. Chem. (1997) 27213, 1849-1855).

During the course of research for the purpose of elucidating the physiological mechanism of the involvement of SCCA in epidermis, the inventors of the present invention surprisingly found that SCCA is an anti-apoptosis factor that has the action of inhibiting cellular apoptosis.

Briefly speaking, as a result of using immunohistological techniques and in situ hybridization to examine the skin's UV defense mechanism focusing on SCCA-1 and SCCA-2, the inventors of the present invention clearly determined that expression of SCCA in sun-exposed sites of skin is remarkably increased as compared with sun-protected sites. Moreover, it was also clearly demonstrated that the expression of SCCA in the spinous layer and granular layer is increased considerably by irradiation of human epidermis with UV light. Protein expression was not observed in the basal layer. In addition, expression of SCCA has been confirmed to be similarly increased by UV radiation in three-dimensional skin models and cultured human keratinocytes.

The inventors of the present invention then established stable expression systems by inserting human SCCA-1 and SCCA-2 genes into 3T3 cells in which expression of SCCA was not observed. As a result of analyzing by fluorescence-activated cell sorting (FACS) using annexin V-FITC and propidium iodine as indicators of apoptosis, apoptosis caused by UV radiation was clearly determined to significantly decrease in both of the stable SCCA expression systems.

Moreover, SCCA-1 and SCCA-2 knockdown (siSCCA) cell lines were established by rendering HaCat cells that highly express SCCA to permanenty express siRNA by means of an RNA interference method using pSilencer vector. Expression of SCCA in the siSCCA cell lines was confirmed to be inhibited by 90% or more by quantitative PCR. As a result of irradiating with UV at 50 mJ/cm², in contrast to 38% of the cells undergoing apoptosis in the control line, roughly 80% of the cells in the siSCCA cell lines were clearly demonstrated to undergo apoptosis.

Next, when skin cells were screened for the presence of interacting factors of SCCA using the antibody array method, the inventors of the present invention found that SCCA interacts with JNK, a stress activated kinase involved in ultraviolet, radiation, oxidative injury and other stress. Moreover, a study of the relationship between SCCA and JNK revealed that SCCA specifically inhibits the kinase activation of JNK-1. JNK-1 is known to induce apoptosis through phosphorylation of transcription factor c-Jun. Thus, SCCA was clearly demonstrated to inhibit apoptosis induced by stress stimuli such as irradiation of cells with ultraviolet light by inhibiting the kinase activity of JNK-1.

The present invention was completed on the basis of the aforementioned findings, and in its first aspect, provides a method for inhibiting the kinase activity of JNK-1 in cells by increasing the expression of SCCA in keratinocytes.

In another of its aspects, the present invention provides a method for treating or preventing diseases caused by activation of JNK-1 by increasing the expression of SCCA in cells and inhibiting kinase activity of JNK-1 in cells. Diseases caused by activation of JNK-1 are preferably skin disorders caused by ultraviolet radiation. In this case, the cells are preferably epidermic cells such as stratum corneum, granule cells and spinous cells.

In still another of its aspects, the present invention provides a pharmaceutical composition or external skin preparation for treating or preventing diseases caused by activation of JNK-1 by inhibiting the kinase activity of JNK-1 in cells, the pharmaceutical composition or external skin preparation containing an amount of SCCA effective for treatment or prevention. Diseases caused by activation of JNK-1 are preferably skin disorders caused by ultraviolet radiation. The cells are preferably epidermic cells such as stratum corneum, granule cells and spinous cells.

According to the present invention, an effective means can be provided for inhibiting stress stimuli and particularly ultraviolet radiation, and more particularly, for treating and preventing skin disorders.

### BRIEF DESCRIPTION OF THE DRAWINGS

FIG. 1 shows the expression of SCCA in epidermis at light-exposed and unexposed sites.

FIG. 2 shows fluctuations in the expression of SCCA in epidermis caused by UV irradiation.

FIG. 3 shows the effects of UV irradiation on SSCA expression in cultured human keratinocytes.

FIG. 4 shows a comparison of apoptosis rates induced by UV irradiation between SCCA highly-expressing cells and SCCA non-expressing cells.

FIG. 5 shows the establishment of an SCCA knockdown cell line.

FIG. 6 shows a comparison of apoptosis rates induced by UV irradiation between SCCA knockdown cells and control cells.

FIG. 7 shows the results of a “pull-down” JNK kinase assay.

### BEST MODE FOR CARRYING OUT THE INVENTION

As was previously described, there have been no reports relating to exogenous inhibitory factors capable of specifically inhibiting JNK only. The ability of SCCA to inhibit its activation caused by stress stimulation of JNK-1 is a surprising fact found for the first time by the inventors of the present invention.

The present invention provides a method for inhibiting the kinase activity of JNK-1 in cells by increasing the expression of squamous cell carcinoma antigen (SCCA) in cells. JNK belongs to the family of MAP kinases that play a central role in information transmission during cell reproduction, differentiation and canceration, and is activated in response to various stress stimuli including ultraviolet irradiation. There are three genes for JNK consisting of JNK-1, JNK-2 and JNK-3. In contrast to JNK-1 inducing apoptosis by being activated by ultraviolet radiation, JNK-2 is not activated by ultraviolet radiation, and the two are known to have different functions. The substrate of JNK-1 in the body is known to be c-Jun. This c-Jun is a transcription factor that induces apoptosis as a result of being phosphorylated by JNK-1. The inventors of the present invention found that SCCA inhibits the phosphorylation of c-Jun by specifically inhibiting the kinase activity of JNK-1, thereby inhibiting the induction of apoptosis by c-Jun.

Thus, it is possible to treat and prevent disorders associated with cellular apoptosis induced by stress stimulation by increasing the expression of SCCA. Examples of this stress stimulation include exposure of the skin to ultraviolet light, various chemical oxidizing agents and atmospheric pollutants, drying stimulation and heat stimulation. Examples of disorders associated with stress stimulation include skin aging caused by cell death due to stress stimulation such as the formation of wrinkles and spots, dry skin, rashes, eczema and burns. The present invention is particularly effective for treating and preventing the aforementioned disorders in subjects poorly exhibiting expression of SCCA.

SCCA is a protein having a molecular weight of about 45,000 that is present in squamous cell carcinoma cells and psoriatic epidermis as previously described. The amino acid sequences of SCCA-1 and SCCA-2 along with the nucleic acid sequences that encode them are described in Takeda A. et al., J. Invest. Dermatol. 118, 147-154 (2002) (op. cit.). An increase in the expression of SCCA-1 and/or SCCA-2 in epidermis can be achieved by, for example, applying a drug that increases their expression.

SCCA-1 and/or SCCA-2 itself may be used for this drug. In this case, SCCA may be naturally-occurring SCCA or recombinant SCCA, and refers to a full-length protein, fragment or other derivative thereof provided that it has an activity of inhibiting JNK-1 kinase activity. Thus, in one of its aspects, the present invention provides a pharmaceutical composition or external skin preparation for treating or preventing diseases due to activation of JNK-1 by inhibiting the kinase activity of JNK-1 in cells, the pharmaceutical composition or external skin preparation comprising an amount of SCCA effective for treatment or prevention. The amount of SCCA effective for treatment or prevention refers to an amount sufficient for inhibiting apoptosis of the cells, and is suitably determined by a physician or veterinarian. Although there are no particular restrictions on this amount, the composition as claimed in the present invention preferably contains 1 μM to 100 mM, more preferably 10 μM to 10 mM, and even more preferably 100 μM to 1 mM SCCA.

In addition, the pharmaceutical composition or external skin preparation as claimed in the present invention can be suitably blended as necessary with ingredients normally used in cosmetics, pharmaceuticals and other external skin preparations in addition to the aforementioned essential ingredient in the form of SCCA or derivative thereof, examples of which include whiteners, moisturizers, antioxidants, oily ingredients, ultraviolet absorbers, surfactants, thickeners, alcohols, powdered ingredients, colorants, aqueous ingredients, water and various types of skin nutrients.

In addition, other ingredients can be suitably blended according to the application of the composition, examples of which include metal chelating agents such as disodium edetate, trisodium edetate, sodium citrate, sodium polyphosphate, sodium metaphosphate and gluconic acid, hot water fruit extracts such as caffeine, tannin, verapamil, tranexamic acid and its derivatives, licorice extract, glabridin and quince, drugs such as various herbal medicines, tocopherol acetate, glycyrrhizic acid and its derivatives or salts, vitamin C, magnesium ascorbic phosphate, glucoside ascorbate, albutin, kojic acid and other whiteners, sugars such as glucose, fructose, mannose, sucrose and trehalose, and vitamins A such as retinoic acid, retinol, retinol acetate and retinol palmitate.

Pharmaceutical or cosmetic compositions as claimed in the present invention may be used in any type of external skin preparation of the prior art according to its application such as cosmetics, pharmaceuticals, over-the-counter medications and other external preparations, examples of which include beauty washes, creams, milky lotions, lotions, facial packs, bath additives, ointments, hair lotions, hair tonics, hair liquids, shampoos, rinses and hair growth preparations, and there are no particular restrictions on the drug form.

When a gene that encodes SCCA in a subject is in an active or silent state, and as a result, cells are in an SCCA-deficient state, the present invention can also be achieved by inserting SCCA-1 and/or SCCA-2 gene itself into epidermic cells or by arranging a regulatory sequence that increases the expression of SCCA-1 and/or SCCA-2 gene, such as a promoter or enhancer, at a location operably-linked to these genes.

Examples of methods that can be applied to insert a gene that encodes SCCA-1 and/or SCCA-2 into cells include gene insertion using a viral vector and non-viral gene insertion methods (Nikkei Science, April 1994, pp. 20-45, Experimental Medicine Supplement, 12(15) (1994); Experimental Medicine Special Issue, “Basic Techniques of Gene Therapy”, Yodosha Publishing (1996)). Examples of gene insertion methods using a viral vector include methods in which genes are inserted by incorporating DNA that encodes SCCA-1 and/or SCCA-2 into a DNA virus or RNA virus such as retrovirus, adenovirus, adeno-associated virus, herpes virus, vaccinia virus, pox virus, polio virus or synbis virus. Methods using retrovirus, adenovirus, adeno-associated virus or vaccinia virus are used particularly preferably. Examples of non-viral gene insertion methods include methods involving direct administration of an expression plasmid (DNA vaccination), liposomes, lipofectin, microinjection, calcium phosphate and electroporation, with the DNA vaccination and liposome methods being used preferably. In addition, in vivo methods, in which DNA is inserted directly into the body, and ex vivo methods, in which a certain type of cells are removed from humans, DNA is inserted into said cells outside the body after which those cells are returned to the body, are used for allowing the aforementioned gene to actually act as a pharmaceutical (Nikkei Science, April 1994, pp. 20-45; Pharmaceuticals Monthly, 36(1), 23-48 (1994); Experimental Medicine Supplement, 12(15) (1994)). In vivo methods are preferable. In the case of being administered by an in vivo method, administration can be carried out by a suitable administration route corresponding to the disease, symptoms and so forth. For example, administration can be carried out by intravenous, intraarterial, subcutaneous, intracutaneous or intramuscular administration. In the case of administering by an in vivo method, the administration is typically in the form of an injection and a commonly used vehicle may be added as necessary. In addition, in the case of using in the form of liposomes or fugogenic liposomes (such as in the case of Sendai virus (HJV)-liposomes), the preparation can be in the form of a liposome preparation such as a suspension, frozen preparation or centrifugally-separated concentrated frozen preparation.

Increased levels of SCCA in cells can be determined by directly measuring the levels of SCCA-1 and/or SCCA-2 in skin cells, for example. Preferably, this measurement is preferably carried out by various methods known in this field such as immunostaining using a fluorescent substance, pigment or enzyme, western blotting or an immunoassay such as ELISA or RIA, using antibody specific for SCCA-1 and/or SCCA-2. In addition, increased levels of SCCA can also be determined by extracting RNA from skin and measuring the amounts of mRNA that encodes SCCA-1 and/or SCCA-2. Extraction of mRNA and measuring of its amounts are carried out by methods known in this field such as RNA quantification by quantitative polymerase chain reaction (PCR). In addition to the above, the expressed amounts of SCCA-1 and/or SCCA-2 can also be measured by measuring the known biological activity of SCCA-1 and/or SCCA-2. In addition, expression of SCCA-1 and/or SCCA-2 can be determined by in situ hybridization and through measurement of biological activity.

The following provides a more detailed explanation of the present invention through its examples. Furthermore, the present invention is not limited to these examples.

Purification of SCCA

Psoriatic scales were homogenized with Tris-buffered saline (TBS) containing 140 mM NaCl, 100 mM Tris-HCl (pH 8.0). The crude homogenate was centrifuged at 16,000× rpm for 60 min. The supernatant obtained was purified using Sephacryl S-200, DEAE Sepharose, Mono Q, Mono S, Mono P and Superose 6 gel chromatography (Pharmacia).

Polyclonal Antibody

A polyclonal antibody to the purified SCCA was raised in New Zealand White rabbits from Kitayama-Rabesu, Japan. Antisera were raised in rabbits by subcutaneous immunization with an emulsified mixture of equal volumes of SCCA (approximately 100 μg per dose) and Freund's complete adjuvant (Statens Seruminstitut, Denmark). The rabbits were boosted at 1 month intervals for three times with SCCA in Freund's incomplete adjuvant. The IgG fraction was purified on a protein A-Sepharose affinity column (Pharmacia) and used for the immunological studies.

Immunohistochemical Examination

Epidermis was biopsied in accordance with the AMeX procedure (Sato Y. et al., Am. J. Pathol., 125, 431-435 (1986)) followed by embedding the specimen in paraffin after fixing with cold acetone. Sections were then removed of paraffin with xylene and then washed with acetone and then PBS. Next, non-specific binding sites of the sections were blocked with 10% normal goat serum (Histofine, Tokyo, Japan).

The epidermal sections were then respectively incubated with anti-SCCA-1 monoclonal antibody (Santa Cruz Biotechnology, California, USA) (diluted to 1:500), anti-SCCA-2 monoclonal antibody (Santa Cruz Biotechnology, California, USA) (diluted to 1:500) or anti-SCCA polyclonal antibody. After washing with PBS, the sections were counter-stained with hematoxylin stain and observed using the Dako Envision System (Dako Corp., California, USA).

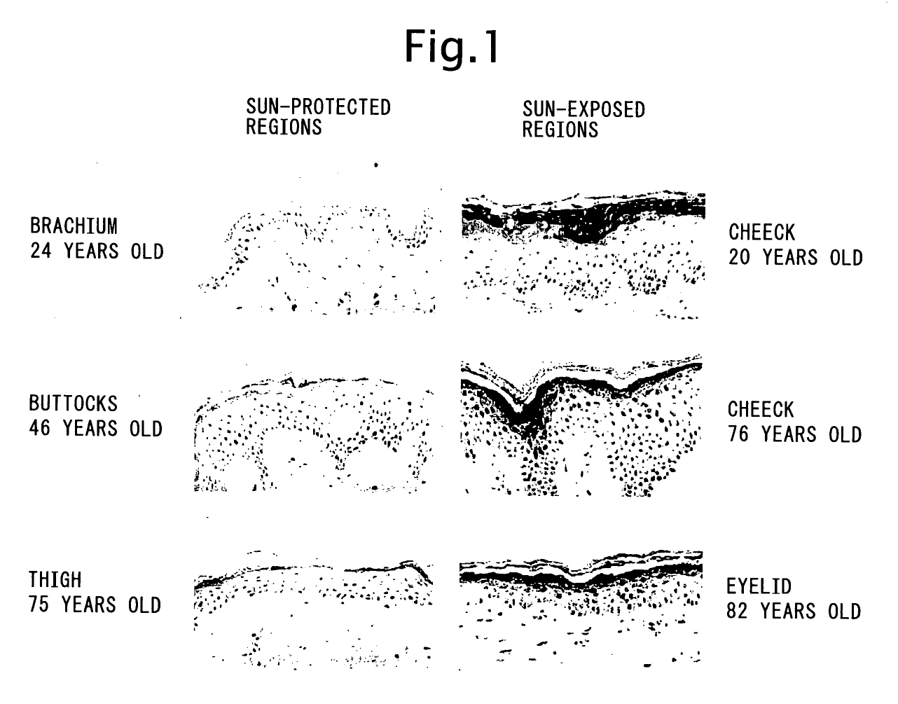

FIG. 1 shows the results of microscopic observations using anti-SCCA polyclonal antibody that binds to both SCCA-1 and SCCA-2 for the antibody after sampling epidermis from sun-protected sites consisting of the brachium (human, 24 years old), buttocks (human, 46 years old) and thigh (human, 75 years old), and epidermis from sun-exposed sites consisting of the cheek (humans, 20 years old, 76 years old) and eyelid (human, 82 years old). It can be understood from FIG. 1 that levels of SCCA are prominently elevated in the upper layer of the epidermis of sun-exposed sites as compared with sun-protected sites. However, an increase in the expression of SCCA was not observed in the basal layer even at the sun-exposed sites.

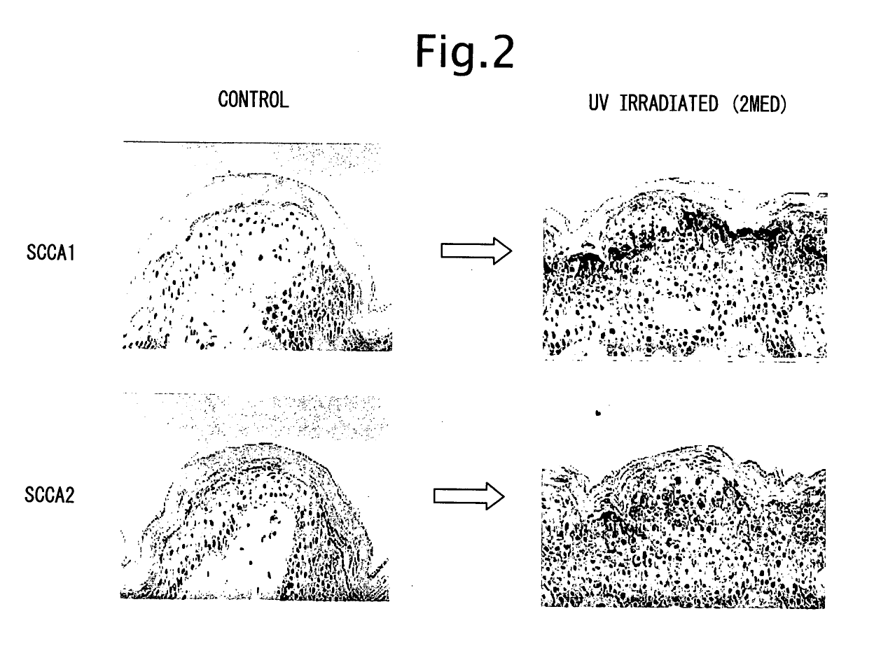

FIG. 2 shows the results of microscopic observations of the respective expression or SCCA-1 and SCCA-2 in human epidermis subjected to irradiation at a dose of 2 MED (minimum erythema dose) using a UV illumination system (TOREX FL205-E-30/DMR Transluminator (Toshiba Medical Supply)), and control epidermis not subjected to irradiation, used for the epidermis specimens. Anti-SCCA-1 monoclonal antibody and anti-SCCA-2 monoclonal antibody were respectively used for the antibodies. It can be understood from FIG. 2 that expression of both SCCA-1 and SCCA-2 was clearly increased by irradiating human epidermis with UV light. In addition, increased expression was prominent in the epidermal spinous layer and granular layer.

According to the above, the expression of SCCA-1 and SCCA-2 in the epidermis, and particularly in its spinous layer and granular layer, was clearly increased when the epidermis was irradiated with UV light.

Quantitative PCR Experiment

Next, an experiment was conducted to confirm at the gene level that expression of SCCA-1 and SCCA-2 in the epidermis is increased by UV irradiation.

Human keratinocytes were cultured at 37° C. in Keratinocyte-SFM medium (Gibco, Invitrogen) in a high humidity atmosphere containing 5% CO₂ in the presence of L-glutamine and epidermic cell growth factor. Cells that had reached a confluence of 60 to 70% were irradiated with UVB for 0 to 48 hours. UVB irradiation was carried out at an intensity of 30 mJ/cm² using the TOREX FL205-E-30/DMR Transluminator (Toshiba Medical Supply). Cells not irradiated with UVB were used for the control cells.

Total RNA was isolated and purified from the aforementioned cultured cells using Isogen (Nippon Gene) in accordance with its instructions. The respective expression levels of SCCA-1 and SCCA-2 were determined by quantitative real-time polymerase chain reaction (PCR). Briefly speaking, total RNA was converted to cDNA using Superscript II (Invitrogen, Carlsbad, Calif., USA). The sample was then amplified by carrying out 40 cycles of 2-step PCR using the ABI PRISM 7900HT Sequence Detection System (Applied Biosystems, Foster City, Calif., USA). G3PDH (glyceraldehyde-3-phosphate dehydrogenase) was used for the internal standard.

The primers that were used are as shown below.

SCCA-1:

| | |
|---|---|
| Forward primer: | |
| 5′-GTGCTATCTGGAGTCCT-3′ | (SEQ. ID NO. 1) |
| Reverse primer: | |
| 5′-CTGTTGTTGCCAGCAA-3′ | (SEQ. ID NO. 2) |
| Taq Man probe: | |
| 5′-CATCACCTACTTCAACT-3′ | (SEQ. ID NO. 3) |

SCCA-2:

| | |
|---|---|
| Forward primer: | |
| 5′-CTCTGCTTCCTCTAGGAACACAG-3′ | (SEQ. ID NO. 4) |
| Reverse primer: | |
| 5′-TGTTGGCGATCTTCAGCTCA-3′ | (SEQ. ID NO. 5) |
| Taq Man probe: | |
| 5′-AGTTCCAGATCACATCGAGTT-3′ | (SEQ. ID NO. 6) |

G3PDH:

| | |
|---|---|
| Forward primer: | |
| 5′-GAAGGTGAAGGTCGGAGTC-3′ | (SEQ. ID NO. 7) |
| Reverse primer: | |
| 5′-GAAGATGGTGATGGGATTTC-3′ | (SEQ. ID NO. 8) |
| Taq Man probe: | |
| 5′-AGGCTGAGAACGGGAAGCTTGT-3′ | (SEQ. ID NO. 9) |

A reporter pigment (6-carboxy-fluoroscein) was bound to the 5′-terminal of the Taq Man probe, and a quencher pigment (6-carboxy-tetramethyl-rhodamine) was incorporated in the 3′-terminal.

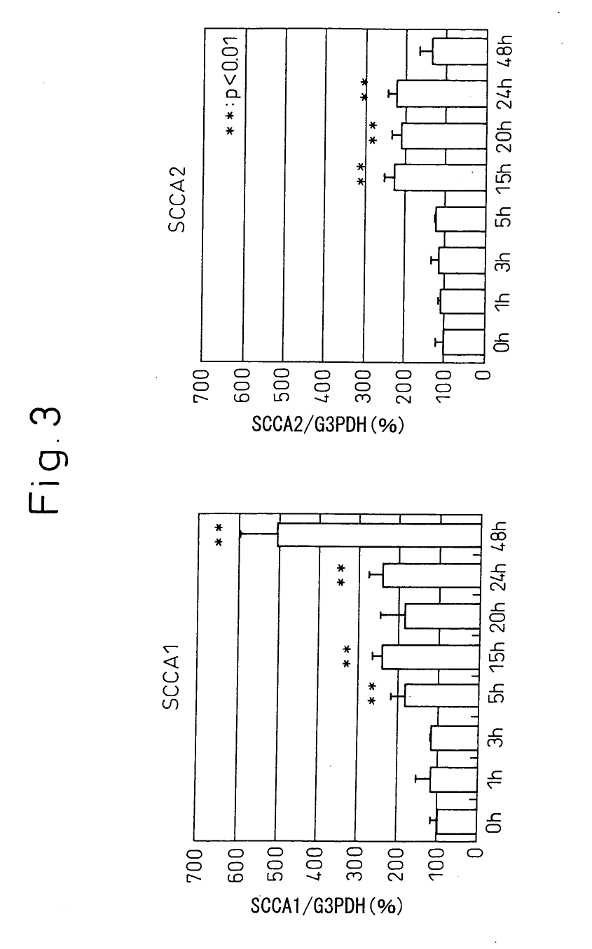

FIG. 3 shows the results of the effects of UVB irradiation on the expression of SCCA in cultured human keratinocytes. The expression of both SCCA-1 and SCCA-2 was clearly increased by UV irradiation. Thus, keratinocytes clearly demonstrated increased expression of SCCA-1 and SCCA-2 at the gene level as a result of UV irradiation.

Examination of the Role of SCCA in UV Irradiation

On the basis of the above, the expression of SCCA-1 and SCCA-2 was clearly demonstrated to increase when subjected to UV irradiation. Next, a study was conducted to examine the roles played by SCCA-1 and SCCA-2 in keratinocytes subjected to UV irradiation.

Establishment of SCCA-1 and SCCA-2 Highly Expressing Cells

3T3 cells (available from ATCC) are cells originating in mouse embryo that do not express SCCA-1 or SCCA-2. A gene that encodes SCCA-1 or SCCA-2 was inserted into these cells in the manner described below.

SCCA-1 and SCCA-2 cDNA (Takeda A. et al., J. Invest. Dermatol. 118, 147-154 (2002)) were digested with Bam HI and Kpn I. These were then subcloned in a pTarget vector and then inserted into 3T3 cells using Lipofectamine Plus (Gibco, Invitrogen Corp.). More specifically, 20 μg of cDNA in 675 μl of serum-free DMEM medium (Invitrogen Corp.) were mixed with 75 μl of Plus reagent and allowed to stand for 15 minutes at 25° C. Lipofectamine (100 μl) was then added to 650 μl of serum-free DMEM medium and this was then added to the aforementioned cDNA-Plus mixture followed by allowing to stand for 15 minutes at 25° C. This cDNA mixture was then added to 10 ml of serum-free DMEM medium and the 3T3 cells were incubated therein for 4 hours in a 5% CO₂ atmosphere at 37° C. This medium was then replaced with DMEM medium containing 10% FCS (Invitrogen Corp.) and incubated overnight. On the following day, G418 (Calbiochem) was added to a final concentration of 500 μg/ml. The concentration of G418 was maintained throughout the culturing period. The medium was replaced every two to three days. After culturing for 4 weeks, several G418-resistant colonies were able to be isolated, and SCCA-1 and SCCA-2 expression cell lines were established.

The cells inserted with cDNA that encodes SCCA-1 (SCCA-1 transfected cells) were confirmed to specifically and stably expressed SCCA-1, and the cells inserted with cDNA that encodes SCCA-2 (SCCA-2 transfected cells) were confirmed to specifically and stably express SCCA-2. In addition, 3T3 cells inserted with a non-specific sequence using the same procedure (control cells) did not express SCCA-1 or SCCA-2.

The roles played by SCCA-1 and SCCA-2 during irradiation with UV light were examined using the aforementioned SCCA-1 inserted cells, SCCA-2 transfected cells and control cells. More specifically, a study was conducted of the roles of SCCA-1 and SCCA-2 on UV-induced apoptosis in epidermis.

Each of the aforementioned cells were cultured at 37° C. in a high humidity, 5% CO₂ atmosphere in DMEM medium containing 10% FCS. Cells that had reached a confluence of 60 to 70% were irradiated with UVB for 0 to 48 hours. UVB irradiation was carried out at an intensity of 50 mJ/cm² using the TOREX FL205-E-30/DMR Transluminator (Toshiba Medical Supply).

Evaluation of apoptosis for these cells was carried out by FACS (fluorescence-activated cell sorting) analysis on the basis of a double-staining method by Annexin V-FITC and propidium iodine (PI) (Annexin V-FITC Kit, Immunotech) using an FACS coulter (EPIX XL-MCL, Beckman Coulter).

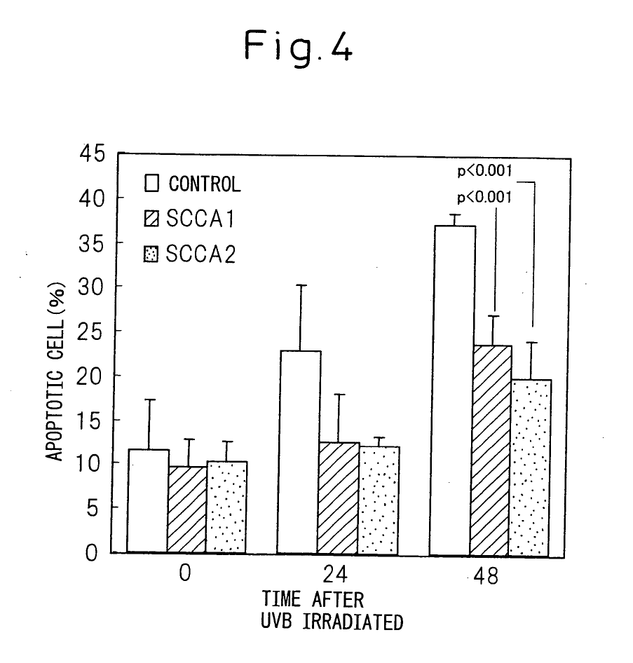

Those results are shown in FIG. 4. As is clear from FIG. 4, apoptosis induced by UV irradiation was observed to significantly decrease in both the SCCA-1 and SCCA-2 transfected cells. Thus, both SCCA-1 and SCCA-2 were presumed to be able to inhibit apoptosis induced by UV irradiation.

In order to confirm this, the inventors of the present invention next additionally examined the roles of SCCA-1 and SCCA-2 in epidermis subjected to UV irradiation by establishing SCCA-1 and SCCA-2 knockdown cell lines by RNA interference.

Establishment of SCCA Knockdown Cells

HaCat cells (H. Hans et al., Experimental Cell Research 239, 339-410 (1998)) are human keratinocytes that express high levels of SCCA. SCCA-1 and SCCA-2 knockdown cell lines were established by rendering these cells to permanently express siRNA (small interference RNA) by means of pSilencer vector (Ambion) in accordance with the RNA interference method.

The siRNA was constructed using the pSilencer vector according to the manual provided. More specifically, a double-strand oligonucleotide, composed of a 65 mer sense oligonucleotide shown below (SEQ. ID NO. 11) containing a 21 mer oligonucleotide (ACATGAACTT GGTGTTGGCT T: SEQ. ID NO. 10) complementary to nucleotide numbers 46 to 66 of a gene that encodes SCCA, and a 65 mer antisense oligonucleotide shown below (SEQ. ID NO. 13) containing a 21 mer oligonucleotide (AAGCCAACAC CAAGTTCATG T: SEQ. ID NO. 12) homologous to nucleotide numbers 46 to 66, was cloned at the Hind III site and Bam HI site of the pSilencer vector. Transfection of HaCat cells was carried out using Lipofectamine 2000 (Invitrogen) according to the manual provided. Control cells were prepared using a double-strand oligonucleotide composed of two oligonucleotides not having significant homology or complementarity with mammalian gene sequences. A stable cell system was acquired by culturing the transfected cells for 4 to 6 weeks in Hygromycin B selective media and selecting the resulting cells. In order to confirm inhibition of the expression of SCCA, the expressed levels of SCCA-1 and SCCA-2 were measured by real-time PCR as previously described.

Sense Oligonucleotide

(SEQ. ID NO. 11)

GATCCCGGCCAACACCAAGTTCATGTTTCAAGAGA
ACATGAACTTGGTGTTGGCTT TTTTGGAAA
(underline indicates complementary region)
Antisense Oligonucleotide

(SEQ. ID NO. 13)

AGCTTTTCCAAAA AAGCCAACACCAAGTTCATGT
TCTCTTGAAACATGAACTTGGTGTTGGCCGG
(underline indicates complementary region)

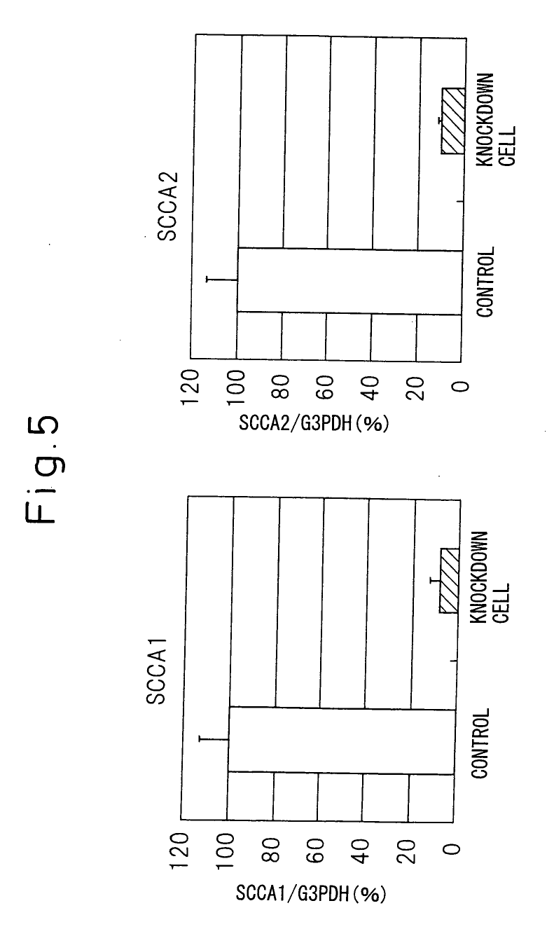

Those results are shown in FIG. 5. The expression of SCCA-1 and SCCA-2 was confirmed to have been inhibited by 90% or more (knockdown) in the cells inserted with the aforementioned siRNA as compared with the control cells.

The roles of SCCA-1 and SCCA-2 against UV-induced apoptosis in epidermic cells were examined using the aforementioned knockdown cells and control cells.

Each of the aforementioned cells were cultured at 37° C. in Keratinocyte-SFM medium (Gibco, Invitrogen) in a high humidity atmosphere containing 5% CO₂ in the presence of L-glutamine and epidermic cell growth factor. Cells that had reached a confluence of 60 to 70% were irradiated with UVB. UVB irradiation was carried out at an intensity of 75 mJ/cm² using the TOREX FL205-E-30/DMR Transluminator (Toshiba Medical Supply).

Evaluation of apoptosis for these cells was carried out by FACS (fluorescence-activated cell sorting) analysis on the basis of double-staining method by Annexin V-FITC and propidium iodine (PI) using an FACS Coulter.

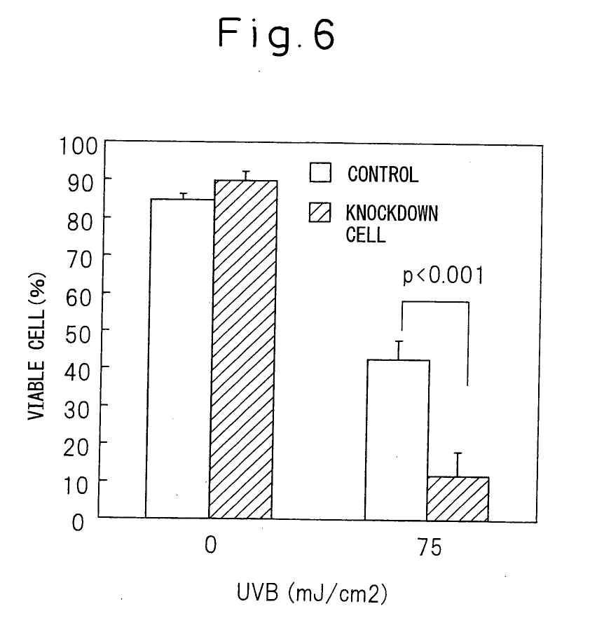

Those results are shown in FIG. 6. As a result of UV irradiation of the knockdown cells, in contrast to apoptosis occurring in 38% of the control cells, apoptosis was clearly induced in roughly 80% of the knockdown cells. Thus, SCCA was determined to significantly inhibit apoptosis of epidermic cells induced by UV irradiation. Accordingly, SCCA is considered to be responsible for the UV defense mechanism of epidermic cells.

Antibody Array

Next, the interaction between SCCA and protein present in epidermic cells was examined to investigate the manner in which SCCA inhibits UV-induced apoptosis.

A Signal Transduction Antibody Array (Hypomatorix, Inc.) was used to screen interactions between proteins. Human oral squamous cell carcinoma line HSC-4 cells (Sumisho-Pharma International), which express high levels of SCCA and were irradiated with UVB at 50 mJ/cm², were dissolved in 1% Triton X-100 extraction buffer containing 15 mM Tris-HCl (pH 7.5), 120 mM NaCl, 25 mM KCl, 2 mM EGTA, 2 mM EDTA, 0.1 mM DTT, 80 μM bestaine and 10 mM peptatin. The insoluble fraction was removed by centrifugal separation at 14,000 rpm for 15 minutes at 4° C. A nitrocellulose membrane immobilized in advance with 400 types of antibodies was incubated while rocking gently in a blocking solution containing 0.1% BSA and 0.1% Tween-20 for 1 hour at room temperature followed by reacting with the aforementioned dissolved cells. After additionally incubating for 2 hours at room temperature, the membrane was washed twice with TBS-T/T (150 mM NaCl, 25 mM Tris-HCl, 0.2% Triton X-100, 0.05% Tween, pH 7.5) and then washed once with TBS after which horseradish peroxidase (HRP)-conjugated anti-SCCA-1 monoclonal antibody or anti-SCCA-2 monoclonal antibody (Santa Cruz Biotechnology, California, USA) bound with was blotted onto the membrane over the course of 2 hours at room temperature. After washing three times with TBS, the membrane was applied to enhanced chemiluminescence (ECL) (Amersham Pharmacia Biotech). As a result, the interesting finding was obtained in which monoclonal antibody to SCCA-1 and SCCA-2 were detected at the location where antibody specific to the active form of stress kinase JNK was spotted, thus indicating that SCCA-1 interacts with active form of JNK. Furthermore, SCCA did not interact with the inactive form of JNK.

Coimmunoprecipitation and Immunoblotting Analysis

The following analyses were carried out to confirm the interaction between SCCA and JNK.

Human oral squamous cell carcinoma line HSC-4 cells, which express high levels of SCCA and were irradiated with UVB at 50 mJ/cm², were dissolved in 1% Triton X-100 extraction buffer containing 15 mM Tris-HCl (pH 7.5), 120 mM NaCl, 25 mM KCl, 2 mM EGTA, 2 mM EDTA, 0.1 mM DTT, 80 μM bestaine and 10 mM peptatin. The insoluble fraction was removed by centrifugal separation at 14,000 rpm for 15 minutes at 4° C. The supernatant was preliminarily purified by continuously shaking for 1 hour at 4° C. with protein G-Sepharose 4B beads. The Protein G-Sepharose 4B beads were removed by centrifuging at 2,000 rpm for 2 minutes at 4° C. The resulting supernatant was then incubated overnight at 4° C. while shaking continuously with Protein G-Sepharose 4B beads bound with anti-SCCA antibody (Santa Cruz Biotechnology) or Protein G-Sepharose 4B beads bound with anti-p-JNK antibody (Cell Signaling and BD Pharmingen). The beads were washed four times with TBS-T/T (150 mM NaCl, 25 mM Tris-HCl, 0.2% Triton X-100, 0.05% Tween, pH 7.5), boiled in 4× sample buffer (20% glycerin, 260 mM Tris-HCl (pH 6.8), 8% SDS) and then electrophoresed on sodium dodecyl sulfate (SDS)-polyacrylamide gel. After transferring to a PVDF membrane, the bound protein was assayed with western blotting using anti-SCCA antibody or anti-p-JNK antibody, respectively. The ECL PLUS Chemiluminescence System (Amersham Pharmacia Biotech) was used for detection. Since p-JNK was detected following incubation with anti-SCCA antibody-bound beads and SCCA was detected following incubation with anti-p-JNK antibody-bound beads, SCCA was confirmed to interact with JNK.

Pull-Down JNK Kinase Assay

(Pull-Down JNK Kinase Assay Using c-Jun-Bound Protein)

As previously described, active JNK-1 is a molecule involved in the transmission of various stress stimuli including ultraviolet light. Active JNK induces apoptosis through phosphorylation of transmission factor c-Jun. Thus, based on the prediction that the anti-apoptotic activity of SCCA is the result of inhibiting the c-Jun phosphorylation activity of JNK, we carried out an in vitro kinase assay for recombinant phosphorylated JNK-1 and recombinant phosphorylated JNK-2 using c-Jun as the substrate in the presence and absence of recombinant SCCA-1.

Recombinant SCCA-1 was produced using a standard method. More specifically, cDNA of SCCA-1 was subcloned to pQE-30 (Qiagen) with a polyhistidine tag (6× His). The His-tagged protein was synthesized in E. coli BL21 (DE31). An Ni-NTA column (Qiagen) and Mono-Q (Amersham) were used to purify the recombinant protein.

2 μg of the aforementioned recombinant SCCA-1, c-Jun fused protein-conjugated beads (Cell Signaling) and 2 μg of active recombinant phosphorylated JNK-1 (Cell Signaling) or 2 μg of active recombinant phosphorylated JNK-2 (Cell Signaling) were incubated overnight at 4° C. while shaking gently. A bead mixture not containing SCCA-1 was incubated as a control. After centrifuging at 2,000 rpm for 2 minutes at 4° C., c-Jun fused beads were washed twice with 500 μl of dissolving buffer (20 mM Tris-HCl (pH 7.4), 150 mM NaCl, 1 mM EDTA, 1 mM EGTA, 1% Triton X-100, 2.5 mM sodium pyrophosphate, 1 mM β-glycerophosphate, 1 mM Na₃VO₄, 1 μg/ml leupeptin and 1 mM PMSF), and then washed twice with 500 μl of kinase buffer (25 mM Tris-HCl (pH 7.5), 5 mM β-glycerophosphate, 2 mM DTT, 0.1 mM Na₃VO₄, 10 MM MgCl₂) followed by carrying out the kinase assay in the manner described below.

Kinase Assay

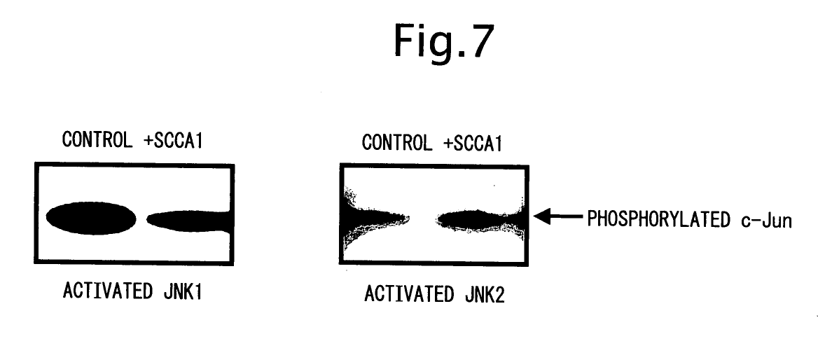

Pellets of c-Jun fused beads were suspended in 50 μl of kinase buffer to which was added 100 μM ATP. After incubating for 30 minutes at 30° C., the reaction product was separated by SDS-polyacrylamide gel electrophoresis and labeled with anti-phosphorylated c-Jun antibody (dilution factor: 1:1000). Those results are shown in FIG. 7. As is clear from the figure, in the case of incubating SCCA-1 with active JNK-1, the amount of phosphorylated c-Jun decreased remarkably as compared with the control not containing SCCA-1. Thus, SCCA-1 was clearly determined to inhibit phosphorylation of c-Jun by JNK-1. On the other hand, in the case of incubating SCCA-1 with active JNK-2, there was no significant decrease in the amount of phosphorylated c-Jun observed as compared with the control. Thus, SCCA-1 was shown to specifically inhibit the activity of JNK-1.

It will be appreciated by those skilled in the art that while the invention has been described above in connection with particular embodiments and examples, the invention is not necessarily so limited and that numerous other embodiments, examples, uses, modifications and departures from the embodiments, examples and use may be made without departing from the inventive scope of this application.

1. A method for inhibiting c-Jun N-terminal kinase 1 (JNK-1) activity in cells by increasing the expression of squamous cell carcinoma antigen (SCCA) in cells.

2. A method for treating or preventing a disease due to activation of JNK-1 by increasing the expression of SCCA in cells, thereby inhibiting kinase activity of JNK-1 in cells.

3. The method according to claim 2 wherein the disease due to activation of JNK-1 is a skin disorder caused by exposure of the skin to ultraviolet radiation.
